(19) United States
(12) Patent Application Publication
(10) Pub. No.: US 2017/0354166 A1
(43) Pub. Date: Dec. 14, 2017

(54) METHOD OF INCREASING LEVELS OF OMEGA-3 FATTY ACIDS IN BEEF PRODUCTS BY ADMINISTRATION OF A GRASS AND ALGAE DIET

(71) Applicant: Donald M Smith, Aspen, CO (US)

(72) Inventor: Donald M Smith, Aspen, CO (US)

(21) Appl. No.: 15/526,955

(22) PCT Filed: Nov. 19, 2015
(86) PCT No.: PCT/US15/61667

§ 371 Date: May 15, 2017

Related U.S. Application Data
(60) Provisional application No. 62/081,760, filed on Nov. 19, 2014.

(51) Int. Cl. A23K 10/16; A23K 20/158; A23K 50/10; A61K 36/05

(52) U.S. Cl. CPC A23K 10/16; A61K 36/05; A23K 20/158; A23K 50/10

(57) ABSTRACT

A method for increasing the levels of omega-3 fatty acids (“omega-3s”), particularly docosahexaenoic acid (DHA) and eicosapentaenoic/icosapentaenoic acid (EPA), in beef products by feeding cattle a diet containing grass as the primary ingredient supplemented with whole algae high in omega-3s. A method of increasing the levels of omega-3s in beef comprising feeding beef animals a daily food ration comprising grass and whole algae.

### BACKGROUND OF THE INVENTION

There is growing consumer demand for beef products high in omega-3 fatty acids (“omega3s”). Existing methods for increasing levels of omega-3s in beef generally consist of feeding cattle whole algae, e.g., Schizochytrium or Nannochloropsis, under typical feedlot conditions. Under these conditions, algae is generally mixed with conventional feedlot ingredients such as grains and harvested forages, e.g., corn, wheat, barley, corn gluten feed, wet or dry distillers grain (DDG) and/or chopped hay.

The efficacy of feeding cattle algae high in polyunsaturated fatty acids (PUFAs) including omega-3s depends on several factors: the strength of the outside cell wall of the algae, the acidity of the environment in the rumen of the animal, the absolute amount of omega-3s in the algae, and the amount of omega-3s in the algae relative to the total fat of the diet (i.e., algae plus feedlot ingredients). To effectively increase the level of omega-3s in cattle, the outside cell wall must remain intact in a large proportion of the algae consumed by the cattle. In effect, the cell wall functions to microencapsulate the PUFAs, including omega-3s, contained in the algae. In the absence of such protection, the PUFAs are exposed to the free hydrogen in the rumen of the cattle, resulting in the biohydrogenation of the PUFAs into saturated fatty acids, i.e., effectively destroying them for the purpose of increasing their presence in the cattle. While the degradation and/or digestion of the cell wall in some portion of algae consumed by the cattle may be inevitable, the more cell walls that survive, the more PUFAs, including omega-3s, are available for absorption in the small intestine of the cattle.

It is well known among ruminant nutritionists that the composition of the diet in cattle determines the degree of acidity in the rumen of the animal, and thus the ability of the ruminal fluid and its contents (e.g., acids, bacteria, etc.) to degrade the cell wall of the algae in the rumen and biohydrogenate the PUFAs, including omega-3s, contained in the algae. The greater the acidity of the rumen, the higher the proportion of the algae in the rumen that will suffer cell wall degradation and consequent PUFA biohydrogenation. Despite the relatively high nutritional quality of beef, it has at times been criticized for the relatively high concentration of saturated fatty acids in the lipid that it contains. These saturated fatty acids are typically found in greater concentrations in beef than in poultry or pork products due to the extensive biohydrogenation that occurs within the rumen. This is the natural process by which the accumulation of organic acids within the rumen impedes the survival and consequent absorption of PUFAs in cattle. The degree of decomposition of algae in the rumen has been measured with varying diets, which themselves produce varying degrees of acidity in the rumen. For example, ruminant nutritionists have calculated that the acidity of a feedlot diet with DDG and without whole corn silage (pH 5.8) is lower than that of a similar feedlot diet with whole corn silage substituted for DDG (pH 5.6).

Cattle typically have a daily food ration which may consist of, e.g., around 20 pounds by dry matter weight of, e.g., grain, grass, corn, sugar beets, etc. It is well known among ruminant nutritionists that cattle will eat their whole daily ration if the fat content of the ration does not exceed approximately 8% by dry matter weight. If the fat content of a given daily ration is about 16%, for example, the cattle will only eat about 50% of the ration. In a typical feedlot ration containing corn (but excluding algae), the corn and other ingredients together produce about 6% fat in the ration. Thus, if an algae ration were to exceed 2% fat when added to the ration, the total ration would contain more than about 8% fat, and the cattle would reduce its daily consumption of the entire feed ration. As a result, the steer would gain less weight and would perform poorly in the feeding program.

There is also increasing production and consumer demand for grass fed beef. To produce grass fed beef, cattle are fed only grass, whether in the pasture and/or in lots. If there is insufficient naturally-occurring grass in the pasture for year-round feeding, the cattle may be fed grass in an enclosure or may be given access to grass (e.g., hay or silage) added to the pasture or to some portion of the pasture.

As defined herein, “grass” is limited to forage consisting of grass (annual and perennial), forbs (e.g., legumes, Brassic), browse, or cereal grain crops in the vegetative (pre-grain) state. As used herein, “grass” for the production of grass fed beef includes, but is not limited to, grass excluding harvested grains or cereal grains in a post-vegetative state; grass meeting the standard set forth in the Grass (Forage) Fed Marketing Claim Standard, Federal Register Notice 72 FR 58631, available at http://www.ams.usda.gov/grades-standards/beef/grassfed; alfalfa cubes, hay, and/or pellets; barley hay; Bermudagrass hay; corn stalk hay; corn, whole hay; forage cubes and/or pellets; grass cubes, hay, and/or pellets; leaves, miscellaneous, dry; legume hay; millet hay; milo stalk hay; milo-soybean hay; mixed mainly grass hay; mixed mainly legume hay; oat hay; OW BLUESTEM; peanut hay; peavine hay; pineapple forage; prairie hay; rice hay; rye hay; small grain hay; sorghum hay; sorghum-Sudan hay; soybean hay; straw and/or straw hay; Sudan hay; Sudangrass hay; sugarcane bagasse, dry; sugarcane hay; sunflower hay; triticale & pea hay; triticale hay; wheat hay and/or straw.

“Grass” further includes, but is not limited to, the following grasses in a pre-grain state: peavine forage; pineapple forage; fresh rice forage; fresh small grain forage; fresh sorghum-Sudan forage; fresh surgarcane; fresh triticale/pea forage; fresh barley forage; fresh Bermuda grass; fresh Brassica forage; fresh browse, miscellaneous; fresh corn silage, forage and/or stalks; fresh grass forage; fresh leaves, miscellaneous; fresh legume forage; fresh millet forage; fresh mixed mostly grass forage; fresh mixed mostly legume forage; fresh oat forage; fresh peanut forage; fresh rye forage; fresh sorghum forage; fresh soybean forage; fresh straw forage; fresh Sudan grass; fresh sugarcane bagasse; fresh sunflower; fresh triticale forage; fresh wheat forage; fresh woody plants; grass pasture; legume pasture; mixed mostly grass pasture; mixed mostly legume pasture; woody plants; oat and/or wheat fodder; barley silage; Bermudagrass silage; corn silage & sunflower; corn stalklage, grass silage; legume silage; millet silage; millet/soybean silage; milo/soybean silage; mixed mainly grass silage; mixed mainly legume silage; oat silage; peanut silage; peavine silage; pineapple forage; processed corn silage; rice silage; rye silage; small grain silage; sorghum silage; sorghum Sudan silage; soybean silage; straw silage; Sudan grass silage; sugarcane bagasse silage; sugarcane silage; sunflower silage; sweet corn silage; Tifton 85 Haylage; triticale silage; triticale/pea silage; wheat silage.

Additional examples of grass are known in the art. For example, sorghum in its vegetative, pre-grain state is a grass. Sudan grass is another non-limiting example of a grass. Similarly, sorghum cut and baled or cut and made into silage before it goes to seed is a grass. However, sorghum allowed to go to seed and harvested as a grain would not meet the definition of “grass” herein.

### SUMMARY OF THE INVENTION

A method for increasing the levels of omega-3 fatty acids (“omega-3s”), particularly docosahexaenoic acid (DHA) and eicosapentaenoic/icosapentaenoic acid (EPA), in beef products by feeding cattle a diet containing grass as the primary ingredient (i.e., more than 70% by weight of food intake), supplemented with whole algae high in omega-3s.

### BRIEF DESCRIPTION OF THE DRAWINGS

FIG. 1 is a graph showing the average weekly dry material intake (DMI) (pounds (lbs) per head per day) of cattle fed a diet of grass supplemented with 1 pound of algae per day (weeks 0-6), 1 pound or greater of algae per day (week 7) or 2 pounds of algae per day (weeks 8-9).

### DESCRIPTION OF THE INVENTION

It has now been discovered that a diet comprising grass supplemented with whole algae, e.g., Schizochytrium or Nannochloropsis, is effective for feeding cattle. Such a diet allows for the production of grass fed beef high in omega-3s, simultaneously addressing two independent consumer demands. It has been discovered that cattle fed with this diet are able to consume more whole algae and thereby produce a beef product containing higher levels of omega-3s than cattle fed using existing methods for increasing levels of omega-3s in beef (i.e., cattle fed whole algae under typical feedlot conditions). Thus, not only does administration of the novel grass and algae diet allow for the production of grass fed beef, the grass fed beef produced is superior to prior art beef high in omega-3s in terms of omega-3 content.

If the cattle are fed only grass, which would entail less than about 2% fat on a 17-25 pound dry matter basis, the cattle will be able to eat more algae with the grass and thereby produce beef containing higher levels of omega-3s. Grass typically contains about 2.0% fat. For example, if the average cow or steer consumes 20 pounds of grass per day (measured as dry material) and if grass on the average contains 2% fat, the cow/steer will have consumed 0.4 pounds of fat. If the cow/steer's total fat intake is limited to approximately 8% of total feed intake per day, this would allow the cow/steer to consume a total of 1.6 pounds of fat per day. An all grass/hay diet contains 0.4 pounds of fat, as described above, leaving an additional 1.2 pounds of fat to be added in the algae. If the algae contains about 60% fat, then feeding the cow/steer 2 pounds of algae will result in the cow/steer consuming 1.2 pounds of fat. Thus, feeding a cow/steer 2 pounds of algae plus 20 pounds of grass per day will result in the cow/steer consuming a total of about 1.6 pounds of fat per day (i.e., the approximate maximum limit).

As another example, some grasses contain only about 1.8% fat, and some cows/steers consume 25 pounds of food per day (measured as dry material). In this case, 8% of 25 pounds of grass is 2 pounds of fat as the daily limit. If the type of grass consumed is 1.8% fat, then the cow/steer will be consuming 0.45 pounds of fat in the grass, leaving 1.55 pounds of fat to be added in the algae. If the algae contains about 60% fat, and if 2.58 pounds of algae are fed daily, the cow/steer will be eating 1.55 pounds of fat from algae plus 0.45 pounds of fat from the grass, or exactly 2.0 pounds of fat (i.e., the approximate maximum limit in this case). Thus, cattle can easily eat 2 to 3 pounds of algae daily, depending on the total amount eaten by the cow/steer, and the percentage of fat in the grass (as well as the percentage of the fat in the algae).

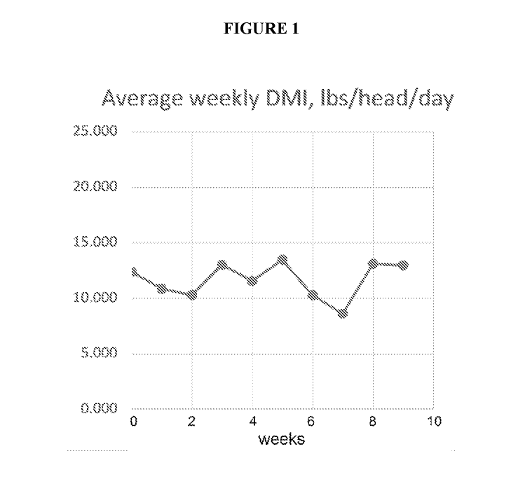

FIG. 1 shows that cattle fed a diet of grass supplemented with a high amount of algae (1 pound or greater, e.g., 2 pounds, per day) are able to maintain a constant amount of feed intake (expressed as dry material intake (DMI)) daily over several (e.g., 9) weeks before slaughter. Cattle were fed a diet of grass supplemented with 1 pound of algae per day in weeks 0-6, and then double the amount (i.e., 2 pounds of algae per day) in weeks 8-9. During week 7 (i.e., the third week before the end of the test), the amount of algae in the diet was gradually increased from 1 pound per day (as in the diet in weeks 0-6) to 2 pounds per day (as in the diet in weeks 8-9). As FIG. 1 shows, the average DMI remained generally constant and did not decrease when the amount of algae in the diet was doubled (from 1 pound per day to 2 pounds per day). Further, the grass fed beef from this experiment was found to contain high levels of omega-3s. The grass fed beef from this experiment contained an average of 63 mg of EPA and DHA per 113 g of meat (ground beef).

In comparison, an article by Smith (Grass-Fed Vs. Grain-Fed Ground Beef—No Difference In Healthfulness, available at http://beefmagazine.com/beef-quality/grass-fed-vs-grain-fed-ground-beef-no-difference-healthfulness) has shown that regular beef (corn-fed) contains approximately 5 mg of DHA plus EPA per 100 g, and regular grass fed beef (without algae) contains approximately 10 mg of DHA plus EPA per 100 g. Both of these are medically insignificant amounts: Mozaffarian et al. (Plasma Phospholipid Long-Chain ω-3 Fatty Acids and Total and Cause-Specific Mortality in Older Adults: A Cohort Study, Ann. Intern. Med. 158 (7): 515-525 (2013), available at http://annals.org/article.aspx?articleid=1671714) has shown that 400 mg of DHA plus EPA daily is very significant medically, and the Canadian Government recommends 500 mg of DHA plus EPA daily for medically beneficial nutrition.

It is anticipated that feeding algae with grass using the method disclosed herein will yield even greater amounts of DHA and EPA per serving, such that a quarter pound hamburger will contain about 200 mg of DHA and EPA and an 8 oz steak will contain about 200 mg DHA plus EPA. Thus, the consumer will be able to consume algae fed grass fed beef and obtain about half the recommended dietary level without eating fish. The consumer will be able to consume additional DHA and EPA by consuming eggs and poultry meat from hens which have been fed the same algae (e.g., Schizochytrium or Nannochloropsis).

Thus, it has been found that if cattle are fed a diet comprising grass plus an amount of whole algae, e.g., Schizochytrium or Nannochloropsis, the steer will eat more of the total feed ration relative to cattle fed whole algae under feedlot conditions, will thus consume more algae, and will produce a beef product containing higher levels of omega-3s including, e.g., DHA and EPA. In experimental tests, for example, cattle fed a diet of grass plus algae consumed two pounds of algae per day, while cattle fed whole algae under feedlot conditions (e.g., rations containing a high corn content) consumed only one pound of algae or less per day.

Algae may be fed to cattle, e.g., in combination with grass. Further, cattle fed algae, e.g., in combination with grass, may be fed one or more additional feeds, optionally combined with grass, as a finishing ration before slaughter. The finishing ration may contain, e.g., high energy ingredients (e.g., potatoes, sugar beets, bagasse, waste, and/or candy products) and/or starches.

Grass or other feeds (e.g., algae and/or finishing rations), optionally in combination, may be fed to cattle, e.g., by grazing or baling or as silage, or by other techniques commonly known in the art.

Food rations fed to cattle are designed to provide a pH in the rumen of between 5.6 and 7.0.

In a preferred embodiment, food rations fed to cattle comprise less than 9% total fat.

In a preferred embodiment, the amount of algae in the total feed ration is between about 0.1 pounds per day and about 3 pounds per day, with the balance of the feed ration being grass, and in some cases additional ingredients as described above to increase caloric intake

In a preferred embodiment, the algae fed to cattle is Schizochytrium.

In a preferred embodiment, cattle are fed a diet comprising between about 0.1 pounds and about 3 pounds of algae, e.g., Schizochytrium, per day, with the balance of the feed ration being grass, and in some cases additional ingredients as described above. If the algae selected has less fats, i.e., lipids, then the amount of algae may be adjusted upward pro rata.

All publications cited herein are incorporated by reference in their entireties.

What is claimed is:

1. A method of increasing the levels of omega-3s in beef comprising feeding beef animals a daily food ration comprising grass and whole algae.

2. The method of claim 1 wherein the beef animals comprise beef cattle.

3. The method of claim 1 wherein the grass comprises more than 70% by weight of the daily food ration.

4. The method of claim 1 wherein the algae is Schizochytrium.

5. The method of claim 1 further comprising feeding the beef animals between about 0.1 and 3.0 pounds per day of algae.

6. The method of claim 1 wherein the grass is fed by grazing or baling or as silage.

7. The method of claim 1 wherein the food ration is designed to provide a pH in the rumen of between 5.6 and 7.0.

8. The method of claim 1 wherein the food ration comprises less than 9% total fat.

9. The method of claim 1 further comprising feeding the beef animals one or more additional feeds as a finishing ration before slaughter.

10. The method of claim 1 wherein the algae comprises Nannochloropsis.
